(19) United States
(12) Patent Application Publication
(10) Pub. No.: US 2015/0174068 A1
(43) Pub. Date: Jun. 25, 2015

(54) Apparatus, Composition, and Related Methods for Transdermal Delivery of Active Ingredients

(71) Applicants: Robert S. Davidson, Woodland Hills, CA (US); Eric Allen, Camarillo, CA (US); Jose Bernado, Thousand Oaks, CA (US); Edward Maliski, Thousand Oaks, CA (US)

(72) Inventors: Robert S. Davidson, Woodland Hills, CA (US); Eric Allen, Camarillo, CA (US); Jose Bernado, Thousand Oaks, CA (US); Edward Maliski, Thousand Oaks, CA (US)

(73) Assignee: CURE IP HOLDINGS, LLC, Oxnard, CA (US)

(21) Appl. No.: 14/344,455

(22) PCT Filed: Sep. 12, 2012
(86) PCT No.: PCT/US12/54795

§ 371 Date: Mar. 13, 2015

Related U.S. Application Data
(60) Provisional application No. 61/533,278, filed on Sep. 12, 2011.

(51) Int. Cl. A61K 9/127; A61K 36/76; A61K 31/455; A61K 31/337; A61K 31/08; A61K 47/24; A61K 31/565; A61K 31/355; A61K 38/28; A61K 47/36; A61K 47/10; A61K 47/46; A61K 31/522; A61K 31/519

(52) U.S. Cl. CPC A61K 9/127; A61K 31/522; A61K 36/76; A61K 31/455; A61K 31/337; A61K 31/08; A61K 31/519; A61K 31/565; A61K 31/355; A61K 38/28; A61K 47/36; A61K 47/10; A61K 47/46; A61K 47/24

(57) ABSTRACT

In accordance with a composition aspect of the invention, a composition for applying to skin comprises one or more pharmaceutically active ingredients contained in a plurality of hydrophobic carriers dispersed throughout a hydrogel, a skin permeation enhancer for enhancing the permeation of the active ingredients into skin, and an emulsifier. The amount of hydrogel is sufficient to allow the hydrogel to dry to as a substantially continuous film over and in contact with the skin to which it is applied. Related method and apparatus aspects are also described.

### CROSS-REFERENCE TO RELATED APPLICATION

This claims the benefit of U.S. provisional application No. 61/533,278, filed Sep. 12, 2011, titled “Apparatus, Composition, And Related Methods for Delivery of Biologically Active Ingredients,” which is incorporated by reference herein in its entirety.

### FIELD OF THE INVENTION

The invention relates to the field of transdermal delivery of active ingredients, and, more particularly, to those including a skin permeation enhancer for increasing absorption by the skin.

### BACKGROUND

Delivery mechanisms for pharmaceutical and nutraceutical active ingredients are a constantly expanding field of research. Most active ingredients are administered in an oral dosage form that is either absorbed through the stomach lining or passed through the stomach where it is absorbed into the bloodstream in the gastrointestinal tract. This pathway presents several drawbacks. Stomach conditions can affect the amount of time it takes for the dose to enter the intestines. The pH of the stomach can also degrade the active ingredient before it becomes available for absorption. Because of these and other drawbacks, the amount of active ingredient that becomes bio-available in the intestines, as well as the time it takes for the active ingredient to enter the intestines, is unpredictable.

An alternative approach is to deliver active ingredients in a dosage form that can be absorbed transdermally through the skin. Conventional transdermal formulations often contain a permeation enhancer that dilates the pores of the skin. By doing so, the permeation enhancer helps the active ingredients in the formulation move beneath the surface of the skin, which enhances absorption of the active ingredients by the body. Although the conventional transdermal delivery formulations are somewhat effective, improvement is needed.

### SUMMARY

Because many conventional permeation enhancers are volatile compounds, we hypothesized that evaporation of the permeation enhancer decreases its effectiveness, which, in turn, diminishes the effectives of the active ingredients. We set out to solve this problem by developing a composition for the transdermal delivery of active ingredients that, when applied to the skin, forms a substantially continuous film over and in contact with the area of skin treated with the composition. The film prevents the permeation enhancer from evaporating, thereby prolonging its effectiveness. Advantageously, the film is formed from one or more of the ingredients in the composition. Accordingly, there is no need for the user to place a bandage or patch over the treated area.

In a composition aspect of the invention, such an advantageous composition comprises: one or more pharmaceutically active ingredients contained in a plurality of hydrophobic carriers dispersed throughout a hydrogel; a skin permeation enhancer for enhancing the permeation of the active ingredients into skin; and an emulsifier. In the composition, the amount of hydrogel is sufficient to allow the hydrogel to dry to as a substantially continuous film over and in contact with the skin to which it is applied.

As shown in the Examples section, the transdermal compositions made with this principle in mind act as an effective delivery system for a variety of active ingredients.

Additional advantages, aspects, and embodiments of the invention are described in the appended Drawings and Detailed Description of Preferred Embodiments.

### BRIEF DESCRIPTION OF THE DRAWINGS

FIG. 1 is a diagram illustrating a phospholipid molecule;

FIG. 2 is a diagram illustrating active ingredients within a micelle;

FIG. 3 is a diagram of a liposome suspended in an aqueous solution;

FIG. 4 is a diagram illustrating a piezoelectric material exposed to expansion and a compression forces and generating a voltage;

FIG. 5 is a side perspective view of a piezoelectric iontophoresis device according to an embodiment of the invention;

FIG. 6 is a diagram illustrating a transdermal composition made in accordance with an embodiment of the invention applied to epidermal tissue;

FIG. 7 is a table that indicates the amount of caffeine collected in Franz-type diffusion cell receptor fluid per time point, calculated as percentage of the applied dose;

FIG. 8 is a table that indicates the cumulative amount of caffeine collected in Franz-type diffusion cell receptor fluid per time point, calculated as percentage of the applied dose;

FIG. 9 is a graph that indicates the cumulative amount of caffeine, nicotinic acid, and paclitaxel collected in Franz-type diffusion cell receptor fluid over a time course;

FIG. 10 is a graph that indicates the cumulative amount of estradiol, polyethylene glycol, and folic acid collected in Franz-type diffusion cell receptor fluid over a time course; and

FIG. 11 is a graph that indicates the cumulative amount of tocopherol acetate and insulin collected in Franz-type diffusion cell receptor fluid over a time course.

### DETAILED DESCRIPTION OF PREFERRED EMBODIMENTS

In the Summary above and in the Detailed Description of Preferred Embodiments, reference is made to particular features (including method steps) of the invention. Where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other ingredients, steps, etc. are optionally present. When reference is made herein to a method comprising two or more defined steps, the steps can be carried out in any order or simultaneously (except where the context excludes that possibility), and the method can include one or more steps which are carried out before any of the defined steps, between two of the defined steps, or after all of the defined steps (except where the context excludes that possibility).

This invention may be embodied in many different forms and should not be construed as limited to the embodiments set forth herein. Rather, these embodiments are provided so that this disclosure will convey preferred embodiments of the invention to those skilled in the art.

### Transdermal Compositions

One of the aspects of the invention is to provide compositions for transdermal delivery of active ingredients that are capable of forming the substantially continuous film on the skin. In the embodiments of the composition, the active ingredients are at least partially contained in hydrophobic carriers that are dispersed in an aqueous medium. Preferably, the hydrophobic carriers are either micelles, liposomes, or oil droplets in a colloidal suspension.

In any of these examples, the aqueous medium preferably includes a gelling agent that is responsible for forming the substantially continuous film when dry. The gelling agent is preferably a hydrocolloid. In preferred embodiments, the hydrocolloid is a hydrogel that forms the aqueous medium around the hydrophobic carriers. Non-limiting examples of hydrocolloids include cellulose derivatives such as hydroxyethylcellulose, methylcellulose, and hydroxypropyl-methylcellulose; agarose, hyaluronan, pectin, pullulan, and other polymers either synthetically or naturally derived. A particularly preferred gelling agent is pectin. A preferred concentration range of gelling agent is approximately 3.7% w/w to approximately 4.1% w/w of the composition.

Aside from the gelling agent, hydrophobic carrier, and active ingredient(s), the transdermal compositions include one or more permeation enhancers to assist the active ingredient(s) in permeating the skin. Examples of permeation enhancers include, but are not limited to, menthyl acetate, DDAIP, fatty acid esters, fatty alcohol ethers, ethanol, dimethylsulfoxide, polyethylene glycol monolaurate, sesquiterpenes, terpenoids, sesquiterpenoids, and terpenes such as menthol, peppermint oil, nerolidol, nerol, linalool, citronellol, and geraniol. A particularly preferred permeation enhancer is menthol. A preferred concentration range of permeation enhancer is approximately 3% w/w to approximately 7% w/w of the composition.

Optionally, the compositions include one or more vasodilators to aid in transdermal active ingredient delivery. Suitable vasodilators include any conventional vasodilator. In a preferred embodiment, the vasodilator is niacin (nicotinic acid). A preferred concentration range of vasodilator is approximately 0.05% w/w to approximately 1% w/w of the composition.

Optionally, the compositions include one or more emulsifiers to prevent the carriers from agglomerating and settling into a continuous oil phase. The use of an emulsifier is more important in the colloidal suspensions. Suitable emulsifiers include, but are not limited to, lecithin, sodium stearyl lactylate, cetearyl alcohol, polysorbates, polyoxyethylene ethers, polyethylene glycol, anisolic compounds, and any conventional emulsifier. Glycerin is a particularly preferred emulsifier due to its relatively low viscosity and reduced propensity to occlude skin pores. A preferred concentration range of emulsifier is approximately 1% w/w to approximately 3% w/w of the composition.

Optionally, the compositions include one or more preservatives for preventing the composition from spoiling. Suitable preservatives include, but are not limited to, antimicrobial preservatives and antioxidants. Examples include sorbic acid and its salts, benzoic acid and its salts, calcium propionate, sodium nitrite, sodium nitrate, sulfites, sulfur dioxide, sodium bisulfite, potassium hydrogen sulfite, disodium EDTA, butylated hydroxyanisole, butylated hydroxytoluene, tert-butylhydroquinone, propyl gallate, ethanol, and methylchloroisothiazolinone. Sodium benzoate is a particularly preferred preservative. A preferred concentration range of preservative is approximately 0.01% w/w to approximately 0.05% w/w of the composition.

As used herein, the term “pharmaceutically active ingredient” or “active ingredient” means an ingredient in the composition that produces a physiological effect in the user. Preferred active ingredients include, but are not limited to, analgesics, anti-inflammatories, stimulants, depressants, sedatives, electrolytes, vitamins, minerals, hormones, peptides, nucleic acids, or any other pharmaceutically active substances,including drugs. A preferred concentration range of active ingredients is approximately 4% w/w to approximately 10% w/w of the composition. However, the concentration range may be expanded or contracted, depending on the particular active ingredient.

The following sections describe more detailed embodiments of the transdermal compositions. Although these detailed embodiments are particularly advantageous, the scope of the invention is not merely limited to these specific embodiments.

In the embodiments of the transdermal composition described below, the active ingredients are caffeine alone or in combination with a source of an alcoholic β-glucoside, an example of which is the analgesic, salicin. White willow bark preferably serves as the source of the alcoholic β-glucoside. A preferred concentration range of caffeine is approximately 4% w/w to approximately 6% w/w of the composition. A preferred concentration range of white willow bark is equal to or less than approximately 4% w/w of the composition.

A. Liposomal Compositions

A transdermal composition, according to an embodiment of the invention, includes micelles and or liposomes dispersed in the aqueous medium. Some preferred substances used to form micelles or liposomes include, but are not limited to, both natural and synthetic phosphatidyl-based substances including lecithins (phosphatidylcholines), hydroxylated lecithin, polyethyleneglycol phospholipid, hydrogenated soy phosphatidylcholine, phosphatidic acid, phosphatidylglycerol, phosphatidylethanolamine, phosphatidylserine, sulfolipids such as sulfoquinovosyl distearoylglycerol, sulfates such as sodium lauryl sulfate, sulfonates such as dioctyl sodium sulfosuccinate, and carboxylates such as sodium deoxycholate, sodium stearate, and sodium oleate.

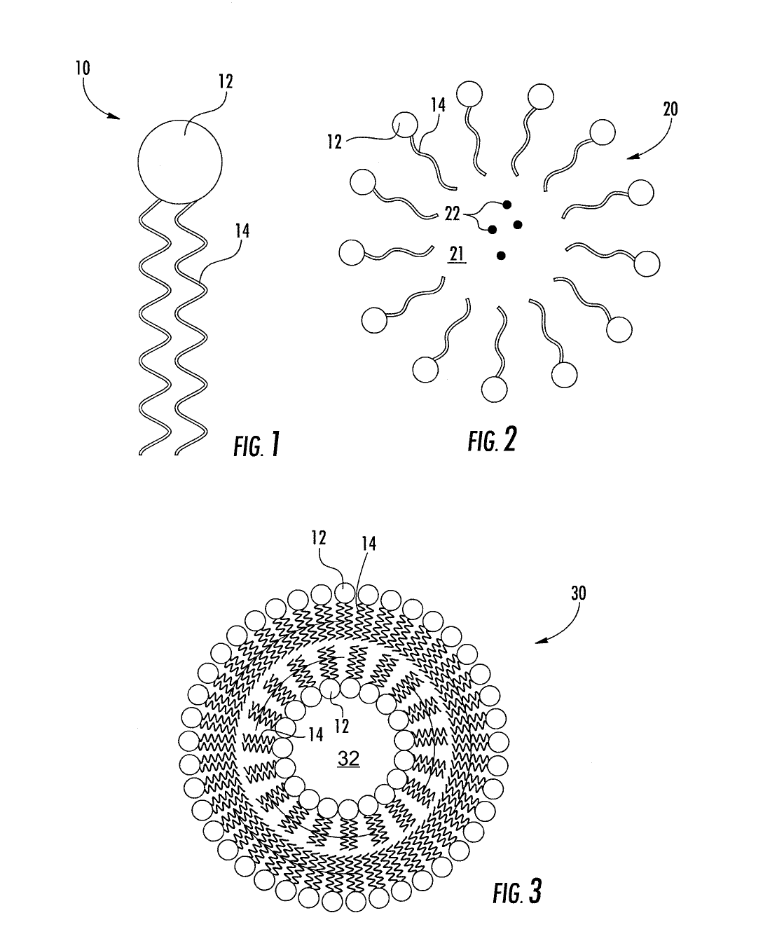

In preferred embodiments, the liposomes and/or micelles are made of one or more phospholipids. Referring to FIG. 1, a phospholipid 10 is a type of amphiphilic lipid. A typical phospholipid 10 has a hydrophilic phosphate head group 12 and a hydrophobic tail 14.

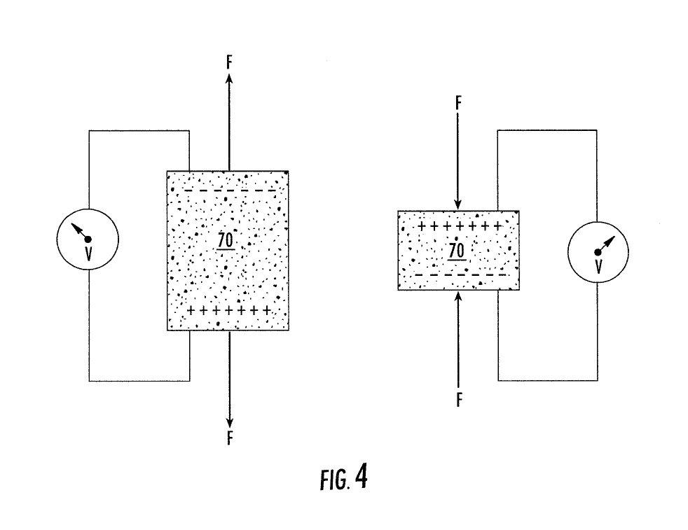

Referring to FIG. 2 a micelle 20 is formed from amphiphilic molecules, such as phospholipids 10. When dispersed in an aqueous solution, the hydrophilic head groups 12 form a hydrophobic pocket 21 composed of the hydrophobic tail groups 14. One or more hydrophobic active ingredients 22 may be encapsulated by the micelle 10 in the hydrophobic pocket 21.

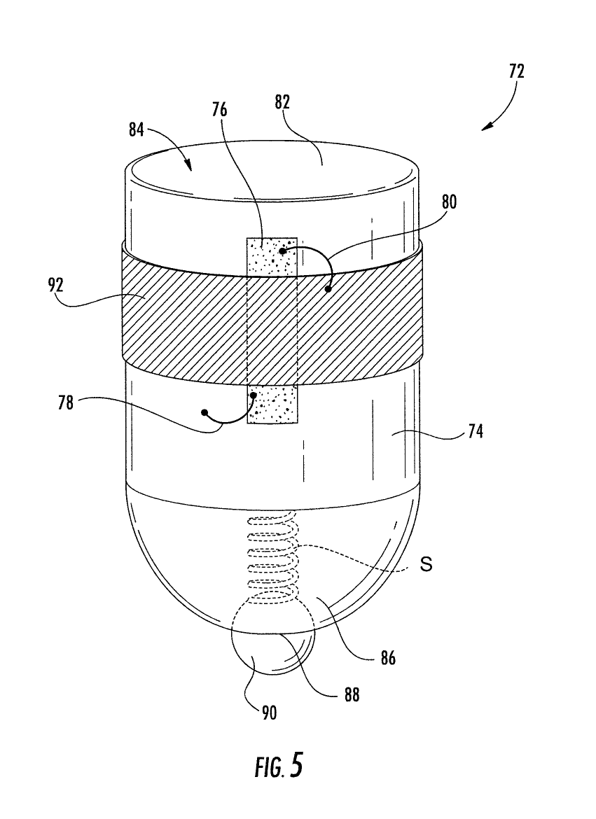

Referring to FIG. 3, a liposome 30 is a vesicle similar to a micelle 20, but composed instead of a plurality of lipid layers. The liposome of FIG. 3 includes a lipid bilayer. Liposomes 30 are capable of encapsulating hydrophobic active ingredients in the hydrophobic tail 14 region of the lipid bilayer and hydrophilic active ingredients in the aqueous pocket 32 at the center of the liposome 30.

Optionally, the surface of the liposomes is charged in order to allow them to be forced into the skin through a process called iontophoresis. In iontophoresis, an electric field is applied to the composition using an electrode placed over the area of the skin that is treated. Negatively charged liposomes are driven into and through the skin by negatively charging the electrode that is in contact with the liposome carriers. Alternatively, positively charged liposomes are driven into and through the skin by positively charging the electrode that is in contact with the liposome carriers.

The charged liposomes for use in iontophoretic delivery are formed by deprotonating a carboxyl group on the phosphate head of the phospholipid used to create the liposomes by reacting the phospholipid with a basic hydroxide salt, such as sodium hydroxide or potassium hydroxide.

A preferred embodiment of the transdermal liposomal composition includes pectin as the gelling agent, menthol as the permeation enhancer, and phosphatidyl serine as the carboxylated phospholipid. We found that when the composition includes approximately 3.7% to approximately 4.1% w/w pectin, approximately 3% to approximately 7% w/w menthol, approximately 1% to approximately 3% w/w glycerin, and approximately 2% to approximately 4% w/w phosphatidyl serine, the composition acts as an effective transdermal delivery system for active ingredients because the viscosity is sufficiently low to allow it to be easily spread over the skin. Further, we found that if the concentration of pectin deviates much out of this range, the desired substantially continuous film will not form. If too little pectin is used, the film is discontinuous on the skin, allowing the permeation enhancer to evaporate. If too much pectin is used, the viscosity of the composition is too high to be easily spread over the skin and the composition is also prone to clogging skin pores.

This liposomal delivery composition is made according to the following method. First, the carboxylated phospholipids are electrically charged by blending the carboxylated phospholipids with the hydroxide salt in an aqueous solution heated to a temperature of approximately 70° C. to approximately 90° C., more preferably approximately 78° C. to approximately 85° C., to form a charged phospholipid solution. The charged phospholipid solution is agitated by shaking, mixing, stirring, or the like with sufficient agitation intensity to open any liposomes in the charged liposome solution. One or more of the desired pharmaceutically active ingredients are then added to the charged phospholipid solution to form a blend. The blend is agitated with sufficient agitation intensity to open any liposomes therein. The agitation intensity is then reduced to allow the liposomes to encapsulate the one or more pharmaceutically active ingredients to form a liposome solution. An emulsifier is added to the liposome solution, then a skin permeation enhancer is added with agitation. The skin permeation enhancer is located external to the liposomes to form an enhanced liposome solution. Pectin is added to the enhanced liposome solution to form the liposomal delivery composition.

In a more detailed embodiment of this method, phosphatidylserine is first dispersed in an aqueous solution to which NaOH is introduced. NaOH deprotonates the carboxyl group of the phosphatidylserine, giving it a negative charge. The aqueous solution of phosphatidylserine and NaOH are mixed at high speed to fully disperse the liposomes throughout the water. This allows the hydroxide ions to interact with all of the liposomes. Preferably, the molar ratio of base:phospholipid is about 1:1 to ensure that each phosphatidylserine molecule is exposed to one hydroxide molecule in order for all, or the majority, of the liposomes to become negatively charged. It is important to first create the negative charge on the liposomes before introducing the active ingredients as the active ingredients may interfere with the deprotonation of the carboxyl group of the phosphatidylserine.

The active ingredients are then added. After the liposomes are formed around the active ingredients the solution is no longer blended at high speed. A high speed blend causes the liposomes to re-open, which will allow the active ingredients to be released from encapsulation.

After the active ingredients are added to the solution, blended, and allowed to rest, glycerin is added. The glycerin helps emulsify the immiscible liquids to evenly distribute solution components throughout the solution. Preferably, in the final composition, at least 70% of the active ingredient(s) is (are) encapsulated in the liposomes. Preferably the viscosity of the final composition is between 45,000 and 100,000 CP at 73° F.

In an apparatus aspect of the invention, an apparatus for applying an electromotive force for use in iontophoretic delivery of active ingredients is provided. Rather than using conventional positive and negative electrodes attached to the skin, however, the apparatus is provided in a more conveniently usable form. The electromotive force is advantageously provided by an apparatus that comprises a piezoelectric member made of a piezoelectric material.

A piezoelectric material is capable of generating electricity when it is exposed to a mechanical force. Referring to FIG. 4, an illustration of how a piezoelectric material 70 generates electricity is shown. When the piezoelectric material 70 is exposed to a force F, positive and negative charges localize on opposite ends of the piezoelectric material 70, generating a voltage. The sign of the voltage changes depending on whether an expansion or compression force F is applied.

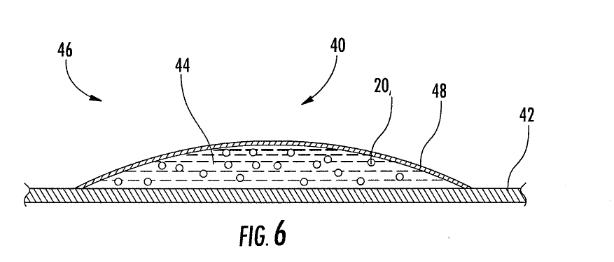

Referring to FIG. 5, in accordance with an apparatus aspect of the invention, a piezoelectric iontophoresis apparatus 72 includes a container 74, a piezoelectric member 76, a first electrical lead 78 and a second electrical lead 80.

The container 74 is preferably made of a compressible material such as a plastic or the like that can contain the transdermal composition. The container 74 includes a first end 82 and a second end 86. The first end 82 is preferably closed and defines a reservoir 84 for holding the composition therein. The second end 86 comprises an opening 88 into which a conductive member 90 is placed. A spring S holds the conductive member 90 in place and also allows the space between the opening 88 and the conductive member 90 to expand by applying downward vertical pressure to the conductive member 90. When this pressure is applied, conductive member 90 retracts slightly into the apparatus 72 creating a larger gap between conductive member 90 and the opening 88, allowing fluid to flow out of the reservoir 84. When the container 74 is positioned with the first end 82 vertical, the composition travels through the reservoir 84 towards the second end 86. The composition exits the second end 86 between the boundary of the opening 88 and the conductive member 90. The conductive member 90 is preferably rotatable within the opening 88 to allow the composition to exit the opening 88 and be applied to the skin of the user via a roll-on type application. Preferably, the conductive member 90 is a metallic ball.

The second electrical lead 80 provides electrical continuity between an affixing member 92 and the piezoelectric member 76. The piezoelectric member 76 preferably includes opposing conductive surfaces to form positive and negative electrodes that contact the first electrical lead 78 and the second electrical lead 80. The first electrical lead 78 is in contact with the conductive member 90 directly by being placed in contact with the composition. In the embodiment shown, the piezoelectric member 76 is affixed to the exterior of the container 74 by the affixing member 92 that is attached to the exterior of the container 74. The piezoelectric member 76 is located between the affixing member 92 and the exterior of the container 74. The affixing member 92 may be wrapped around the container 74 or adhered to the container 74. In this manner, the container label is conveniently used as the affixing member 92. The affixing member 92 is preferably at least partially made of a conductive material which contacts the second electrical lead 80. The type of affixing member 90, however, is not intended to be a limiting feature of the apparatus. Affixing members such as tapes or adhesives, may be used alone or in combination with other types of affixing members 90.

When a user intends to apply the composition, the user places the conductive member 90 against his or her skin and squeezes the container 74 and piezoelectric member 76. Squeezing the container 74 forces the composition out of the reservoir 84 onto the skin. Squeezing the piezoelectric member 76 generates a voltage. The second electrical lead 80 is preferably in contact with the squeezing hand and fingers through the conductive affixing member 92. The voltage created provides an electromotive force between the conductive member 90 and the user's skin, which causes the charged entities in the composition to permeate the skin when contacted by the conductive member 90.

In an alternative embodiment of the apparatus (not shown), the container includes a plunger located on the first end. When the user presses the plunger towards the reservoir, the resulting pressure pushes the composition out of the opening on the second end. Pressing the plunger also places pressure on the piezoelectric member to create an electromotive force. As with the embodiment of FIG. 5, the negative electrode of the piezoelectric member is electrically connected to the conductive member via a brush or preferably an electrode immersed in the composition within the container.

### Colloidal Compositions

In embodiments in which the transdermal composition comprises a colloidal suspension, the hydrophobic carriers are lipophilic particles or droplets suspended in the aqueous medium. Such hydrophobic carriers can be formed from almond oil, argan oil, avocado oil, canola oil, cashew oil, castor oil, coconut oil, cod liver oil, colza oil, corn oil, cottonseed oil, fish oil, grapeseed oil, hazelnut oil, hemp oil, linseed oil (flaxseed oil), macadamia oil, marula oil, mongongo nut oil, mustard oil, olive oil, palm oil (palm kernel oil), peanut oil, pecan oil, perilla oil, pine nut oil, pistachio oil, poppy seed oil, pumpkin seed oil, grapeseed oil, rice bran oil, safflower oil, sesame oil, soybean oil, sunflower oil, tea seed oil, walnut oil, watermelon seed oil, and combinations thereof. Grapeseed oil is particularly preferred due to its relatively low viscosity.

Preferably, the diameter of the suspended hydrophobic carriers is small enough to pass through pores of human skin, which are typically about 50-90 μm in diameter.

We found that when the composition comprises 3.7%-4.1% w/w pectin, 3%-7% w/w menthol, 1%-3% w/w glycerin, and 1%-3% w/w grapeseed oil, each being combined with one or more pharmaceutically active ingredients in the aqueous medium, the substantially continuous film formed on the user's skin and the diameter of the suspended hydrophobic carriers were between approximately 10 μm to approximately 50 μm.

This colloidal delivery composition is made according to the method that is now described. Grapeseed oil and glycerin are mixed in an aqueous solution heated to a temperature of approximately 70° C. to approximately 90° C. or, more preferably, approximately 78° C. to approximately 85° C. The one or more pharmaceutically active ingredients, menthol, and pectin are then added to the mixture to form a blend. The blend is finally agitated until it becomes homogeneous.

Preferably, the viscosity of the colloidal delivery composition is between 45,000 and 100,000 CP at 73° F. or, more preferably, about 90,000 CP at 73° F. The pH of the final composition is preferably about 4.

In light of the discussion regarding the liposomal and colloidal compositions, it should be noted that the hydrophobic carrier does not necessarily need to be either oil droplets or liposomes. Embodiments of the invention include compositions in which the both are used as hydrophobic carriers.

### Use of the Transdermal Compositions

The transdermal compositions are preferably designed to be administered topically in the form of a gel. They may be administered with or without the aid of a delivery device.

Referring to FIG. 6, a transdermal composition is illustrated atop the skin. In this illustrative example, the composition 40 is applied to the epidermis 42. As discussed above, the composition 40 includes hydrophobic carriers 20 dispersed in the aqueous medium 44. When the composition is applied to the epidermis 42, the gelling agent dries to form a substantially continuous film 48. The film 48 prevents the permeation enhancer from evaporating, thereby extending the duration of time during which the permeation enhancer is effective. This provides a greater opportunity for active ingredients to be absorbed.

In a method of treatment aspect of the invention, a method of treating a physiological condition in a subject comprises contacting the skin of the subject with at least one transdermal composition made in accordance with a composition aspect of the invention. Suitable subjects include humans and animals.

Contacting may be accomplished by depositing the composition onto the skin and rubbing the composition over the surface of the area of skin that the user intends to treat. This can be done by hand or through the use of an applicator. In a preferred method of contacting, the user dispenses the composition from a container equipped with a roller-ball applicator and spreads the composition over the area to be treated with the roller-ball. This technique is advantageous because it evenly distributes the composition over the skin.

The term “physiological condition” refers to a bodily condition that the subject desires to treat. These include, for example, diseases, illnesses, pain, chemical imbalances, tiredness, alertness, sleeplessness, and dehydration among many others.

### EXAMPLES

This section describes several examples of more specific transdermal compositions and methods for making them. It also presents experimental data highlighting the effectiveness of the transdermal compositions for transdermal delivery of active ingredients. These examples do not limit the scope of the various aspects and embodiments of the invention in any way.

### Example 1

### Liposomal Composition

A liposomal composition, in accordance with a composition aspect of the invention, is provided in Table 1. This exemplary composition includes the active ingredients caffeine and white willow bark, which are liposomally encapsulated. With these active ingredients, the composition functions as a topical transdermal stimulant and analgesic delivery vehicle.

TABLE 1

| Ingredient | % by weight(w/w) |
|---|---|
| Water, Purified USP | 81.03% |
| Genu Pectin | 3.95% |
| Phosphatidylserine | 2.00% |
| Glycerin 99.7% USP | 2.00% |
| Sodium Hydroxide | 0.20% |
| Caffeine Anhydrous, USP | 5.00% |
| Menthol Crystals BP/USP | 3.80% |
| White Willow Bark | 2.00% |
| Sodium Benzoate | 0.02% |
| Total: | 100.00% |

The composition of Table 1 was prepared according to the following procedure. Purified USP water was heated to approximately 78-85° C. (172-185° F.) in a heating tank. The water was charged to a mixing container, and blended using a high-shear variable speed mixer at 1000 rpm. While blending, phosphatidylserine was charged to the mixing container.

Still blending at the same speed, sodium hydroxide was charged to the mixing container. This solution was mixed at 3600 rpm for 2-3 minutes. The speed of the mixing was then returned to 1000 rpm while the anhydrous caffeine and white willow bark were added to solution. The solution was then blended at 3600 rpm until the appearance of homogeneity was reached.

The mixing was halted, and the solution rested for about 5-7 minutes, allowing the solution to slightly cool. The mixing was resumed at 1000 rpm and the glycerin was added to the solution. Then the mixing was sped up to 2000 rpm until the appearance of homogeneity was reached.

The solution was then mixed at 1000 rpm, and menthol and sodium benzoate were added, in that order. Once all of these were added to the solution, the speed of mixing was increased to 2000 rpm and blended until the appearance of homogeneity was reached.

Mixing was continued at 1000 rpm and the pectin was added. Mixing speed was increased to 2000 rpm and the pectin was added to the solution and blended until the appearance of homogeneity was reached.

### Example 2

### Colloidal Composition

A colloidal composition, in accordance with a composition aspect of the invention, is provided in Table 2. This exemplary composition includes the active ingredients caffeine and white willow bark in a colloidal suspension. With these active ingredients, the composition functions as a topical transdermal stimulant and analgesic delivery vehicle.

TABLE 2

| Ingredient | % by weight(w/w) |
|---|---|
| Water, Purified USP | 81.17% |
| Genu Pectin | 3.95% |
| Glycerin 99.7% USP | 2.00% |
| Grapeseed Oil | 2.00% |
| Caffeine Anhydrous, USP | 5.00% |
| Menthol Crystals BP/USP | 3.80% |
| White Willow Bark | 2.00% |
| Niacin | 0.05% |
| Potassium Chloride | 0.01% |
| Sodium Benzoate | 0.02% |
| Total: | 100.00% |

The composition of Table 2 was prepared according to the following procedure. Purified USP water was heated to approximately 78-85° C. (172-185° F.) in a heating tank. The water was charged to a mixing container, and blended using a high-shear variable speed mixer at 3100 rpm. While blending, grapeseed oil and glycerin were charged to the mixing container and mixed for about 1-2 minutes, until the solution appeared homogeneous.

Still blending at the same speed, anhydrous caffeine, niacin, white willow bark, menthol crystals, and sodium benzoate were added to the solution, in that order. This solution was mixed at 3100 rpm for 2-3 minutes, until the appearance of homogeneity was reached.

The solution was then mixed at 2200 rpm, and pectin was added and blended until the appearance of homogeneity was reached.

### Example 3

### In Vitro Transdermal Caffeine Delivery Studies

Pre-clinical selection of topical formulations was performed using an in-vitro penetration study through human epidermal membrane, which assesses the percutaneous absorption profiles of applied drugs. The method is based on the use of excised human skin as a membrane in a diffusion system that simulates in-vivo skin conditions. By analyzing the amount of the drug reaching the receptor solution, the penetration profile of the drug in the skin can be assessed and used as a tool to determine the feasibility of formulations for subsequent development.

Embodiments of the transdermal compositions were tested in an in vitro penetration study through human epidermis as a testing membrane to simulate topical drug delivery. In particular, excised human epidermis was utilized to assess the penetration profiles of caffeine.

Two transdermal compositions were tested: Formulation A and Formulation B. Formulation A included 79.37% Purified USP water, 3.95% Genu Pectin 150 USA-SAG TYPE D Slow Set, 2.00% USP Glycerin (99.7%), 2.00% Grapeseed Oil, 5.00% USP Anhydrous Caffeine, 7.60% BP/USP Menthol Crystals, 0.05% USP Niacin, 0.01% Potassium Chloride, and 0.02% Sodium Benzoate. Formulation B included 73.67% Purified USP water, 3.95% Genu Pectin 150 USA-SAG TYPE D Slow Set, 2.00% USP Glycerin (99.7%), 2.00% Grapeseed Oil, 5.00% USP Anhydrous Caffeine, 1.90% BP/USP Menthol Crystals, 0.05% USP Niacin, 0.01% Potassium Chloride, and 0.02% Sodium Benzoate.

Formulation A was tested with and without occlusion. Parafilm was applied for occlusion tests, and the samples were kept under occlusion during the period of the study. Radiolabeled [8-14C]-caffeine was incorporated in each formulation prior to the application, and used to analyze the samples. The penetration profile was assessed by collecting receptor fluid samples at various time-points from a chamber positioned below the epidermal samples. The selected time-points were 3 minutes, 10 minutes, 1 hour and 2 hours after the application of the formulations to the surface of the epidermal membrane.

The results showed Formulation A delivered the most caffeine at all time-points. Occlusion using Parafilm decreased the amount of caffeine penetrating across the epidermis. Formulation B yielded the least caffeine at the end of study

The materials for the assays were: [8-14C]-caffeine in ethanol solution under argon (Catalog# MC-499, Lot No. 195-132-0521-A-20100630-MW, Specific activity: 52.1 mCi/mmol; concentration 0.1 mCi/mL; 375.9 μg/ml); Formulation A; and Formulation B. Each formulation was mixed with radioactive caffeine which was dried overnight from 1 mL solution per 2 mL formulation. Therefore, each formulation contained radioactive caffeine of 50 μCi/mL. The mixtures were stored at a 4° C. refrigerator after preparation. The skin used in the study was obtained from abdominal surgery on a single human donor, who provided written consent.

An open label, within donor, comparison study of topical formulations was performed. The formulations were tested on epidermal membrane using a finite dose application. Each formulation was applied to the surface of the epidermal membranes at a target dose of 15.6 μL/cm2 (=10 μL/cell). The receptor solution was continuously stirred, bathing the lower surface of the membrane. At the time-points noted above, receptor fluid samples were collected by draining the receptor chamber. The chamber was fully replenished with fresh receptor fluid. The amount of radioactive caffeine was analyzed from the receptor fluid samples.

The conditions tested were Formulation A, Formulation A—with epidermal surface occlusion, and Formulation B.

The in vitro delivery study was performed using Franz diffusion cells. The diffusion cells were mounted on a holder with magnetic stirrer. The water jacket compartment of each cell was connected to a circulating water-bath. The temperature of the cells was maintained by flowing water from the water-bath controlled at 33° C. The human epidermal membrane was mounted between the donor chamber (where the formulation is applied) and the receptor chamber. The receptor chamber was filled with phosphate buffered saline (pH 7.4; 1 mM) as receptor liquid. The delivery through human epidermis was assessed for 2 hours after the application of the formulations. The receptor fluid was collected in a vial from the receptor chamber of the diffusion cell at 4 time-points: 3 minutes, 10 minutes, 1 hour, and 2 hours. The vial was weighed and saved for sample analysis. The skin membranes were stored. From the results of the analysis, the amount of caffeine penetrating through human epidermis was determined.

Excised skin was obtained from human donor, fresh (within 24 hour after harvest) or frozen (stored within 24 hour after harvest, in temperature <4° C. for not more than 3 days, or in temperature <−20° C. for not more than 1 week). The donors were not younger than 16 years of age and not older than 70 years of age, and were absent disease conditions affecting the normal appearance of skin. The skin was harvested mainly, but not exclusively from, the abdomen, back or thigh. During transport, the harvested skin was placed in media such as MEM (Eagle's Minimum Essential Media) or equivalents.

For skin preparation, after the removal of subcutaneous fat, the surface of the skin is wiped clean with tissue paper soaked with purified water. The skin was placed, dermal side down, on hot plate set at 60° C. for approximately 2 minutes. The epidermis was then separated from dermis. The epidermis sheet is washed twice in purified water, then air-dried and stored at room temperature prior to use. Epidermis sheet from a single donor is cut into multiple smaller membrane sections to fit on the Franz-type diffusion cells (Permegear Inc., Bethlehem, Pa.). The receptor solution of Phosphate Buffered Solution (PBS pH 7.4, 1 mM) fills the receptor chamber of the diffusion cell, free from any air bubbles and continuously bathes the inner surface of the membrane. The donor chamber of the cell is left open to the ambient laboratory environment. Each cell is equipped with water jacket connected to a circulating water bath maintained at 33.0±0.2° C.

Formulations were applied in triplicate to sections of the same donor skin at a target dose of 5-20 μL/cell (or 7-35 mg/cm2). The formulations were applied and distributed to the skin using a positive displacement pipette.

The receptor fluid was collected in a vial at the specified time-points from the receptor chamber of the diffusion cell. Fresh receptor fluid refilled the receptor chamber. The vial was weighed and saved. The receptor fluid was collected up to the specified end-of-study time.

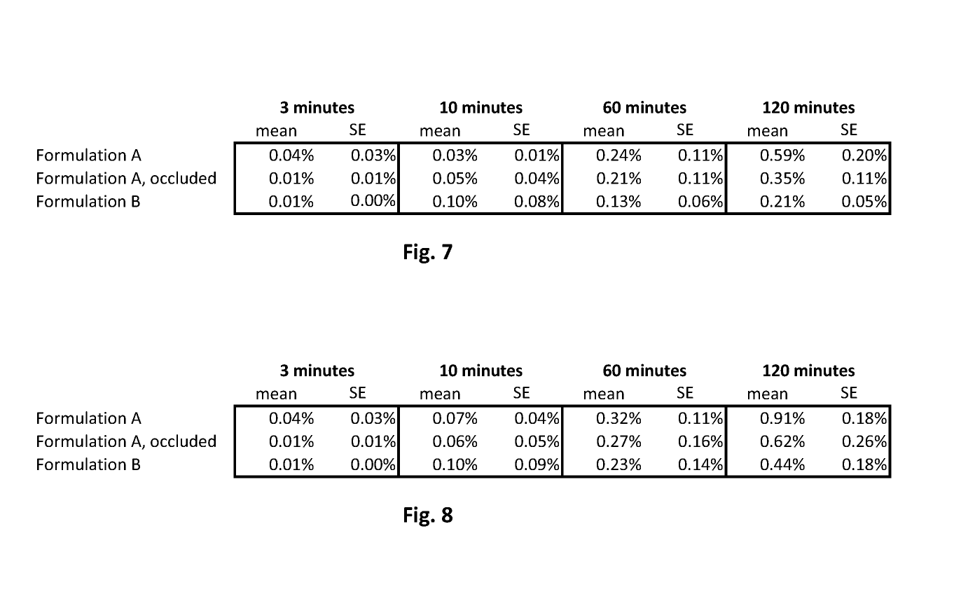

FIG. 7 indicates the amount of caffeine collected in the Franz-type diffusion cell receptor fluid per time point, calculated as percentage of the applied dose. Caffeine penetrated through the epidermis into the receptor chamber within the first 3 minutes, increasing in the amount at each time point. One of six replicates of Formulation B application had a higher penetration rate at the 10-minute time-point, which was considered to be an anomaly of the biological membranes. Application of Formulation A and Formulation B-with occlusion, except for the 10-minute time-point, yields higher amounts of caffeine penetrating the epidermis than the application of Formulation B. Occlusion is shown to reduce the penetration of caffeine through the epidermis from the same formulation, comparing Formulation A to Formulation A-with occlusion.

FIG. 8 indicates the cumulative amount of caffeine collected in the Franz-type diffusion cell receptor fluid per time point, calculated as percentage of the applied dose. At the 120-minute (2-hour) time-point, the cumulative amount of caffeine penetrating from Formulation A application was the highest. The cumulative amount of caffeine from Formulation A application is twice as much as the one from Formulation B application. As already observed from the time-point data of FIG. 7, occlusion reduced the penetration of caffeine through the epidermis from the Formulation A application, followed by Formulation A-with occlusion and the least was Formulation B. Measureable penetration of caffeine across human epidermis was observed already in the first 3 minutes after the application of formulations. This is the first reported in vitro study to pass caffeine through the epidermis in three minutes.

Formulation A yields the highest amount of caffeine penetrating through the epidermis, whereas Formulation B yields the lowest. Formulation A with higher menthol content is related to higher caffeine penetration across the epidermis. Occlusion with Parafilm reduced the caffeine penetration across the epidermis.

### Example 4

### In Vitro Transdermal Delivery Studies of Various Active Ingredients

Nine transdermal compositions were tested. Caffeine was liposomally encapsulated in a composition designated “Proprietary Encapsulation I.” Eight additional compositions were tested in a colloidal composition designated “Proprietary Encapsulation II,” each composition having a different active ingredient. The active ingredients tested were caffeine, nicotinic acid, paclitaxel, estradiol, polyethylene glycol, folic acid, tocopherol acetate, and insulin.

Proprietary Encapsulation I included a liposomal mixture comprising 78.56% Purified USP water, 3.80% Genu Pectin 150 USA-SAG TYPE D Slow Set, 2.00% USP Glycerin (99.7%), 0.50% Grapeseed Oil, 2.00% Hydroxylated Soy Lecithin, 5.00% USP Anhydrous Caffeine, 7.60% BP/USP Menthol Crystals, 0.50% USP Niacin, 0.02% Potassium Sorbate, and 0.02% Sodium Benzoate.

Proprietary Encapsulation II included a colloidal mixture of 5.00% active ingredient in 79.06%, Purified USP water, 3.8% Genu Pectin 150 USA-SAG TYPE D Slow Set, 2.00% USP Glycerin (99.7%), 2.00% Grapeseed Oil, 7.60% BP/USP Menthol Crystals, 0.5% USP Niacin, 0.02% Potassium Sorbate, and 0.02% Sodium Benzoate.

Radiolabeled active ingredient was incorporated in each formulation prior to the application, and used to analyze the samples. The penetration profile was assessed by collecting receptor fluid samples at various time-points from a chamber positioned below the epidermal samples. The time-points were taken over a 24 hour period after the application of the formulations to the surface of the epidermal membrane.

Each formulation was mixed with a radioactive active ingredient which was dried overnight to contain radioactive active ingredient of 50 μCi/mL. The mixtures were stored at 4° C. after preparation. The skin used in the study was obtained from abdominal surgery on a single human donor, who provided written consent.

An open label, within donor, comparison study of topical formulations was performed. The formulations were tested on epidermal membrane using a finite dose application. Each formulation was applied to the surface of the epidermal membranes at a target dose of 15.6 μL/cm2 (=10 μL/cell). The receptor solution was continuously stirred, bathing the lower surface of the membrane. At various recorded time-points, receptor fluid samples were collected by draining the receptor chamber. The chamber was fully replenished with fresh receptor fluid. The amount of radioactive active ingredient was analyzed from the receptor fluid samples.

The in vitro delivery study was performed using Franz diffusion cells. The diffusion cells were mounted on a holder with magnetic stirrer. The water jacket compartment of each cell was connected to a circulating water bath. The temperature of the cells was maintained by flowing water from the water-bath controlled at 33° C. The human epidermal membrane was mounted between the donor chamber (where the formulation is applied) and the receptor chamber. The receptor chamber was filled with phosphate buffered saline (pH 7.4; 1 mM) as receptor liquid. The delivery through human epidermis was assessed for 2 hours after the application of the formulations. The receptor fluid was collected in a vial from the receptor chamber of the diffusion cell at seven recorded time-points: 3 minutes, 1 hour, 2, 3.5, 5, 8 and 24 hours post-application. The vial was weighed and saved for sample analysis. The skin membranes were stored. From the results of the analysis, the amount of active ingredient penetrating through human epidermis was determined.

Excised skin was obtained from human donor, fresh (within 24 hour after harvest) or frozen (stored within 24 hour after harvest, in temperature <4° C. for not more than 3 days, or in temperature <−20° C. for not more than 1 week). The donors were not younger than 16 years of age and not older than 70 years of age, and were absent disease conditions affecting the normal appearance of skin. The skin was harvested mainly, but not exclusively from, the abdomen, back or thigh. During transport, the harvested skin was placed in media such as MEM (Eagle's Minimum Essential Media) or equivalents.

For skin preparation, after the removal of subcutaneous fat, the surface of the skin was wiped clean with tissue paper soaked with purified water. The skin was placed, dermal side down, on hot plate set at 60° C. for approximately 2 minutes. The epidermis was then separated from dermis. The epidermis sheet was washed twice in purified water, then air-dried and stored at room temperature prior to use. Epidermis sheet from a single donor was cut into multiple smaller membrane sections to fit on the Franz-type diffusion cells (Permegear Inc., Bethlehem, Pa.). The receptor solution of Phosphate Buffered Solution (PBS pH 7.4, 1 mM) fills the receptor chamber of the diffusion cell, free from any air bubbles and continuously bathes the inner surface of the membrane. The donor chamber of the cell was left open to the ambient laboratory environment. Each cell was equipped with water jacket connected to a circulating water bath maintained at 33.0±0.2° C.

Formulations were applied in triplicate to sections of the same donor skin at a target dose of 5-20 μL/cell (or 7-35 mg/cm2). The formulations were applied and distributed to the skin using a positive displacement pipette. Additional replicate formulation applications are planned.

The receptor fluid was collected in a vial at the specified time-points from the receptor chamber of the diffusion cell. Fresh receptor fluid refilled the receptor chamber. The vial was weighed and saved. The receptor fluid was collected up to the specified end-of-study time.

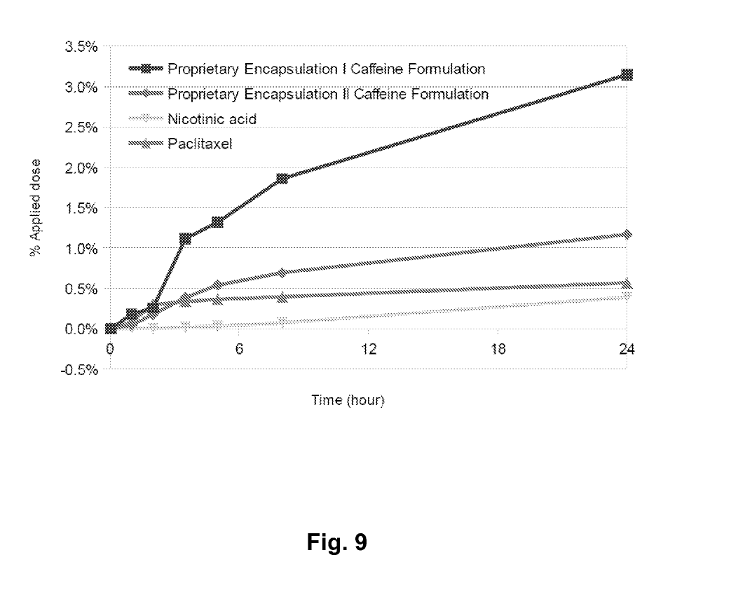

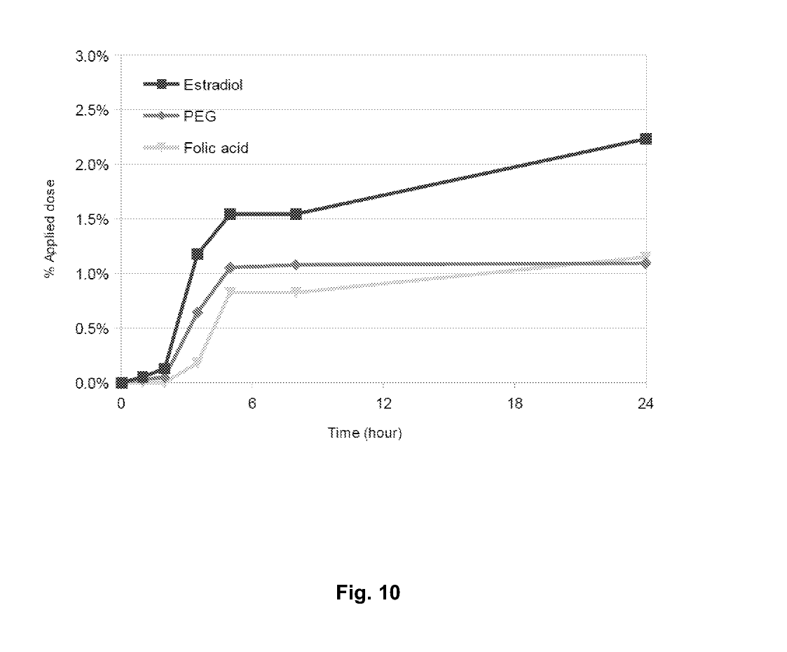

FIG. 9 illustrates the amount of caffeine, nicotinic acid, or Paclitaxel passing through the epidermis. Caffeine was in “Proprietary Encapsulation I” or “Proprietary Encapsulation II.” Greater than 3% of the caffeine penetrated the epidermis in the Proprietary Encapsulation II over 24 hours, while greater than 1% of the caffeine penetrated the epidermis in the Proprietary Encapsulation I over 24 hours. Nicotinic acid and Paclitaxel were each suspended in Proprietary Encapsulation I, and approximately 0.5% of these active ingredients, independently, penetrated the epidermis over 24 hours.

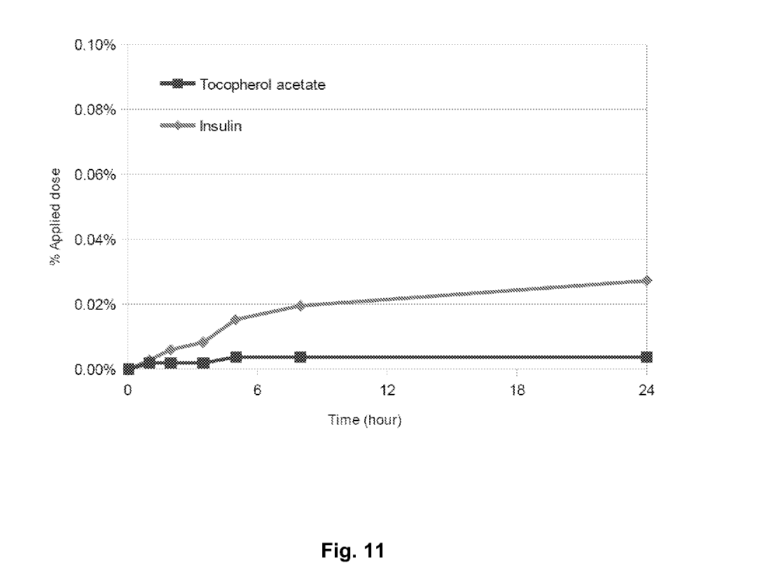

FIG. 10 illustrates the amount of estradiol, polyethylene glycol, and folic acid that passed through the epidermis. These active ingredients were also encapsulated in colloidal Proprietary Encapsulation II. Approximately 1% of polyethylene glycol and folic acid each penetrated the epidermis over 24 hours. Greater than 2% of the estradiol each, independently, penetrated the epidermis over 24 hours.

FIG. 11 illustrates the amount of tocopherol acetate and insulin passing through the epidermis. These active ingredients were also in colloidal Proprietary Encapsulation II. Trace amounts of tocopherol acetate penetrated the epidermis over 24 hours. Greater than 0.02% of the insulin penetrated the epidermis over 24 hours.

The invention has been described with reference to preferred embodiments. Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the invention, suitable methods and materials are described. The skilled should understand that the methods and materials used and described are examples and may not be the only ones suitable for use in the invention.

Various modifications and changes can be made within the spirit and scope of the invention as described in the foregoing specification and as defined in the appended claims.

1. A composition for applying to skin, the composition comprising:
one or more pharmaceutically active ingredients contained in a plurality of hydrophobic carriers dispersed throughout a hydrogel;
a skin permeation enhancer for enhancing the permeation of the active ingredients into skin; and
an emulsifier;
wherein the amount of hydrogel is sufficient to allow the hydrogel to dry to as a substantially continuous film over and in contact with the skin to which it is applied.

2. The composition of claim 1, wherein the hydrophobic carriers comprise oil and are approximately 1% to approximately 3% w/w of the composition, the hydrogel is approximately 3.7% to approximately 4.1% w/w of the composition, the emulsifier is approximately 1% to approximately 3% w/w of the composition, and the skin permeation enhancer is approximately 3% to approximately 7% w/w of the composition.

3. The composition of claim 2, wherein the oil comprises grapeseed oil, the hydrogel comprises pectin, the emulsifier comprises glycerin, and the skin permeation enhancer comprises menthol.

4. The composition of claim 3, wherein the one or more pharmaceutically active ingredients are approximately 4% to approximately 10% w/w of the composition.

5. The composition of claim 4, wherein the one or more pharmaceutically active ingredients include caffeine, the caffeine being approximately 4% to about 6% w/w of the composition.

6. The composition of claim 5, wherein the one or more pharmaceutically active ingredients further include white willow bark, the white willow bark being equal to or less than approximately 4% w/w of the composition.

7. The composition of claim 2, wherein the one or more pharmaceutically active ingredients are approximately 4% to approximately 10% w/w of the composition.

8. The composition of claim 7, wherein the one or more pharmaceutically active ingredients include caffeine, the caffeine being approximately 4% to approximately 6% w/w of the composition.

9. The composition of claim 8, wherein the one or more pharmaceutically active ingredients further include white willow bark, the white willow bark being equal to or less than approximately 4% w/w of the composition.

10. The composition of claim 2, wherein the one or more pharmaceutically active ingredients include white willow bark, the white willow bark being equal to or less than approximately 4% w/w of the composition.

11. The composition of claim 1, wherein the hydrophobic carriers comprise phospholipid-based liposomes and are approximately 1% to approximately 3% w/w of the composition, the hydrogel is approximately 3.7% to approximately 4.1% w/w of the composition, the emulsifier is approximately 1% to approximately 3% w/w of the composition, and the skin permeation enhancer is approximately 3% to approximately 7% w/w of the composition.

12. The composition of claim 11, further comprising a hydroxide salt in approximately a 1:1 molar ratio with the phospholipids.

13. The composition of claim 1, wherein the hydrophobic carriers comprise phospholipid-based liposomes and are approximately 1% to approximately 3% w/w of the composition, the hydrogel is approximately 3.7% to approximately 4.1% w/w of the composition, the emulsifier is approximately 1% to approximately 3% w/w of the composition, and the skin permeation enhancer is approximately 3% to approximately 7% w/w of the composition.

14. The composition of claim 13, wherein the phospholipid-based liposomes comprise phosphatidyl serine, the hydrogel comprises pectin, the emulsifier comprises glycerin, and the skin permeation enhancer comprises menthol.

15. The composition of claim 13, wherein the one or more pharmaceutically active ingredients are approximately 4% to approximately 10% w/w of the composition.

16. The composition of claim 15, wherein the one or more pharmaceutically active ingredients include caffeine, the caffeine being approximately 4% to approximately 6% w/w of the composition.

17. The composition of claim 16, wherein the one or more pharmaceutically active ingredients further include white willow bark, the white willow bark being equal to or less than approximately 4% w/w of the composition.

18. The composition of claim 13, wherein the one or more pharmaceutically active ingredients are approximately 4% to approximately 10% w/w of the composition.

19. The composition of claim 18, wherein the one or more pharmaceutically active ingredients include caffeine, the caffeine being approximately 4% to approximately 6% w/w of the composition.

20. The composition of claim 19, wherein the one or more pharmaceutically active ingredients further include white willow bark, the white willow bark being equal to or less than approximately 4% w/w of the composition.

21. The composition of claim 13, wherein the one or more pharmaceutically active ingredients include white willow bark, the white willow bark being equal to or less than approximately 4% w/w of the composition.

22. A method of treating a physiological condition in a subject, the method comprising contacting the skin of the subject with the composition of claim 1.

23. A composition comprising 3.7%-4.1% w/w pectin, 3%-7% w/w menthol, 1%-3% w/w glycerin, and 1%-3% w/w grapeseed oil, each combined with one or more pharmaceutically active ingredients in an aqueous medium.

24. The composition of claim 23, wherein the one or more pharmaceutically active ingredients are 4%-10% w/w of the composition.

25. The composition of claim 23, wherein the one or more pharmaceutically active ingredients include caffeine, the caffeine being 4%-6% w/w of the composition.

26. The composition of claim 25, wherein the one or more pharmaceutically active ingredients further include white willow bark, the white willow bark being equal to or less than 4% w/w of the composition.

27. The composition of claim 23, wherein the one or more pharmaceutically active ingredients include white willow bark, the white willow bark being equal to or less than 4% w/w of the composition.

28. The composition of claim 23, further comprising niacin, the niacin being 0.05%-1% w/w of the composition.

29. The composition of claim 23, further comprising niacin, the niacin being 0.05%-1% w/w of the composition and wherein the one or more pharmaceutically active ingredients include caffeine and white willow bark, the caffeine being 4%-6% w/w of the composition, the white willow bark being equal to or less than 4% w/w of the composition.

30. A method of treating a physiological condition in a subject, the method comprising contacting the skin of the subject with the composition of claim 23.

31. A method of making a topical composition, the method comprising:
mixing grapeseed oil and glycerin with an aqueous solution heated to a temperature of approximately 70° C. to approximately 90° C.;
adding one or more pharmaceutically active ingredients, menthol, and pectin to the mixture to form a blend; and
agitating the blend until it becomes homogeneous;
wherein the blend comprises 3.7%-4.1% w/w pectin, 3%-7% w/w menthol, 1%-3% w/w glycerin, and 1%-3% w/w grapeseed oil.

32. The method of claim 31, wherein the one or more pharmaceutically active ingredients are 4%-10% w/w of the blend.

33. The method of claim 32, wherein the one or more pharmaceutically active ingredients include caffeine, the caffeine being 4%-6% w/w of the blend.

34. The method of claim 33, wherein the one or more pharmaceutically active ingredients further include white willow bark, the white willow bark being equal to or less than 4% w/w of the blend.

35. The method of claim 31, wherein the one or more pharmaceutically active ingredients includes white willow bark, the white willow bark being equal to or less than 4% w/w of the blend.

36. A composition comprising 3.7%-4.1% w/w pectin, 3%-7% w/w menthol, 1%-3% w/w glycerin, and 2%-4% w/w phosphatidyl serine, each combined with one or more pharmaceutically active ingredients in an aqueous medium.

37. The composition of claim 36, further comprising sodium hydroxide in approximately a 1:1 molar ratio with phosphatidyl serine.

38. The composition of claim 36, wherein the one or more pharmaceutically active ingredients are 4%-10% w/w of the composition.

39. The composition of claim 38, wherein the one or more pharmaceutically active ingredients include caffeine, the caffeine being 4%-6% w/w of the composition.

40. The composition of claim 39, wherein the one or more pharmaceutically active ingredients further include white willow bark, the white willow bark being equal to or less than 4% w/w of the composition.

41. The composition of claim 36, wherein the one or more pharmaceutically active ingredients include white willow bark, the white willow bark being equal to or less than 4% w/w of the composition.

42. The composition of claim 36, further comprising niacin, the niacin being 0.05%-1% w/w of the composition.

43. The composition of claim 36, further comprising sodium hydroxide in approximately a 1:1 molar ratio with phosphatidyl serine, and wherein the one or more pharmaceutically active ingredients include caffeine and white willow bark, the caffeine being 4%-6% w/w of the composition, the white willow bark being equal to or less than 4% w/w of the composition.

44. A method of treating a physiological condition in a subject, the method comprising contacting the skin of the subject with the composition of claim 36.

45. A method of making a topical composition, the method comprising:
electrically charging carboxylated phospholipids by blending the carboxylated phospholipids with a hydroxide salt in an aqueous solution heated to a temperature of approximately 70° C. to approximately 90° C. to form a charged phospholipid solution;
agitating the charged phospholipid solution with sufficient agitation intensity to open any liposomes therein;
adding one or more pharmaceutically active ingredients to the charged phospholipid solution to form a blend;
agitating the blend with sufficient agitation intensity to open any liposomes in the blend;
reducing the agitation intensity and waiting for a time sufficient to allow liposomes to encapsulate the one or more pharmaceutically active ingredients to form a liposome solution;
blending an emulsifier with the liposome solution;
adding a skin permeation enhancer to the liposome solution with agitation, the skin permeation enhancer being located external to the liposomes to form an enhanced liposome solution; and
adding pectin to the enhanced liposome solution to form a topical composition.

46. The method of claim 45, wherein the molar ratio of carboxylated phospholipids to hydroxide salt is about 1:1.

47. The method of claim 46, wherein the hydroxide salt is sodium hydroxide.

48. The method of claim 45, wherein the carboxylated phospholipid includes phosphatidyl serine, the emulsifier includes glycerin, and the skin permeation enhancer includes menthol.

49. The method of claim 48, wherein the topical composition comprises 3.7%-4.1% w/w pectin, 3%-7% w/w menthol, 1%-3% w/w glycerin, and 2%-4% w/w phosphatidyl serine.

50. The method of claim 48, wherein the topical composition further comprises sodium hydroxide in approximately a 1:1 molar ratio with phosphatidyl serine.

51. The method of claim 48, wherein the topical composition comprises 3.7%-4.1% w/w pectin, 3%-7% w/w menthol, 1%-3% w/w glycerin, 2%-4% w/w phosphatidyl serine, 4%-6% w/w caffeine, and less than or equal to 4% w/w white willow bark.

52. The method of claim 45, wherein the one or more pharmaceutically active ingredients are 4%-10% w/w of the topical composition.

53. The method of claim 52, wherein the one or more pharmaceutically active ingredients include caffeine, the caffeine being 4%-6% w/w of the topical composition.

54. The method of claim 53, wherein the one or more pharmaceutically active ingredients further include white willow bark, the white willow bark being equal to or less than 4% w/w of the topical composition.

55. The method of claim 52, wherein the one or more pharmaceutically active ingredients includes white willow bark, the white willow bark being equal to or less than 4% w/w of the topical composition.

56. A transdermal formulation for topical administration, comprising:
at least one active ingredient, wherein the at least one active ingredient is contained in a plurality of hydrophobic carriers dispersed in an aqueous medium containing a hydrocolloid, wherein the amount of aqueous medium is sufficient to create a substantially continuous film over the applied area;
a skin permeation enhancer for enhancing the permeation of the active ingredient(s) into the skin;
a vasodilator; and
an emulsifier.

57. The formulation of claim 56, wherein the at least one active ingredient is approximately 4% to approximately 10% (w/w) of the formulation.

58. The formulation of claim 56, wherein the active ingredient is selected from the group consisting of caffeine, white willow bark, nicotinic acid, paclitaxel, estradiol, polyethylene glycol, folic acid, tocopherol acetate, insulin, and combinations thereof.

59. The formulation of claim 56, wherein the active ingredient is caffeine.

60. The formulation of claim 59, wherein measurable sub-epidermal penetration of the caffeine occurs within about 3 minutes after administration.

61. The formulation of claim 56, wherein the active ingredients are caffeine and nicotinic acid.

62. The formulation of claim 61, wherein measurable sub-epidermal penetration of the caffeine occurs within about 3 minutes after administration.

63. The formulation of claim 56, wherein the active ingredient is polyethylene glycol.

64. The formulation of claim 63, wherein approximately 1% of the polyethylene glycol penetrates the epidermis within 24 hours after administration.

65. The formulation of claim 56, wherein the active ingredient is folic acid.

66. The formulation of claim 65, wherein approximately 1% of the folic acid penetrates the epidermis within 24 hours after administration.

67. The formulation of claim 56, wherein the active ingredient is estradiol.

68. The formulation of claim 67, wherein greater than 2% of the estradiol penetrates the epidermis within 24 hours after administration.

69. The formulation of claim 56, wherein the active ingredient is tocopherol acetate.

70. The formulation of claim 56, wherein the active ingredient is insulin.

71. The formulation of claim 70, wherein greater than 0.02% of the insulin penetrates the epidermis within 24 hours after administration.

72. The formulation of claim 56, wherein the hydrophobic carriers comprise oil.

73. The formulation of claim 59, wherein the oil is grapeseed oil.

74. The formulation of claim 56, wherein the hydrophobic carriers are approximately 1% to approximately 3% (w/w) of the formulation.

75. The formulation of claim 56, wherein the emulsifier is selected from the group consisting of lecithin, sodium stearoyl lactylate, cetearyl alcohol, polysorbates, polyoxyethylene ethers, polyethylene glycol, anisolic compounds, and glycerine.

76. The formulation of claim 56, wherein the emulsifier is glycerine.

77. The formulation of claim 56, wherein the emulsifier is approximately 1% to approximately 3% (w/w) of the formulation.

78. The formulation of claim 56, wherein the skin permeation enhancer is selected from the group consisting of menthol, menthyl acetate, DDAIP, fatty acid esters, fatty alcohol ethers, ethanol, dimethylsulfoxide, polyethylene glycol monolaurate, sesquiterpenes, terpenoids, sesquiterpenoids, and terpenes.

79. The formulation of claim 56, wherein the skin permeation enhancer is menthol.

80. The formulation of claim 56, wherein the skin permeation enhancer is approximately 3% to approximately 7% (w/w) of the formulation.

81. The formulation of claim 56, wherein the hydrocolloid is selected from the group consisting of hydroxyethylcellulose, methylcellulose, hydroxypropyl-methylcellulose, agarose, hyaluronan, pectin, and pullulan.

82. The formulation of claim 56, wherein the hydrocolloid is pectin.

83. The formulation of claim 56, wherein the vasodilator is nicotinic acid.

84. The formulation of claim 56, wherein the vasodilator is approximately 3% to approximately 5% (w/w) of the formulation.
